(19) United States
(12) Patent Application Publication
(10) Pub. No.: US 2008/0208163 A1
(43) Pub. Date: Aug. 28, 2008

(54) Use of Reverse Thermosensitive Polymers to Control Biological Fluid Flow Following a Medical Procedure

(75) Inventor: James A. Wilkie, Melrose, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Pluromed, Inc., Woburn, MA (US)

(21) Appl. No.: 12/035,703

(22) Filed: Feb. 22, 2008

Related U.S. Application Data
(60) Provisional application No. 60/902,817, filed on Feb. 22, 2007.

(51) Int. Cl. A61B 17/03; A61M 31/00

(52) U.S. Cl. 604/506; 606/213

(57) ABSTRACT

One aspect of the present invention relates to a method to control biological fluid flow at a site in a mammal by use of an in situ formed polymer plug. In certain embodiments, the present invention relates to a method to control bleeding following a catheterization procedure, a method to control leakage of cerebral spinal fluid following a lumbar puncture, a method to seal a fistula, or a method to control the flow of serous fluid after a lymphadenectomy. In certain embodiments, the polymer plug is generated in situ by temperature changes, pH changes or ionic interactions. In certain embodiments, the polymer plug comprises at least one optionally purified reverse thermosensitive polymer.

### RELATED APPLICATIONS

This application claims the benefit of priority to U.S. Provisional Patent Application Ser. No. 60/902,817, filed Feb. 22, 2007; the entirety of which is hereby incorporated by reference.

### BACKGROUND OF THE INVENTION

There is a need to close punctured arteries after peripheral arterial catheterization procedures. A variety of methods are being used, from manual pressure to biological devices to complex mechanical devices. For example, complex mechanical devices include Starclose from Abbott Laboratories.

One of the widely used “plug” methods involves the use of absorbable collagen plugs, in particular to close the femoral arterial puncture site after cardiac catheterization under full anticoagulation. A potential complication of this method is acute ischemia in the lower leg. Steil and co-workers have observed acute ischemia after successful closure of the puncture site with VasoSeal in the right lower leg of 2% of patients. Angiography confirmed acute occlusion of the distal A. poplitea dextra. A 25-mm resp. 50 mm-long cylindrical foreign body embolus was removed with a Fogarty-catheter by retrograde indirect embolectomy. Histopathology confirmed a fresh collagen clot with appositional thrombosis. (Stiel, G. M. et al. Z. Kardiol. 1992, 81(10), 543-5.)

Unfortunately, previous attempts to use water soluble reverse thermosensitive polymers for such arterial closure have failed, largely because the presence of an introducer prevented any effective occlusion effect. Specifically, previous work has shown that one can obtain cessation of intra-renal blood flow using a 22% solution of poloxamer 407, which forms a solid gel at 19° C. (J. Raymond, A. Metcalfe, I. Salazkin, and A. Schwarz, “Temporary vascular occlusion with poloxamer 407,” Biomaterials 2004, 25, 3983.) However, this polymer was developed for a different purpose, namely hemostasis in smaller and cooler surface-exposed arteries, and it was found that while catheters could be retrieved from femoral arteries, for example, without any compression or bleeding when poloxamer was used for closure, after about 15-30 minutes the wound would suddenly reopen in all cases, necessitating routine compression for haemostasis.

In contrast to the previous reports in the literature, one aspect of the present invention remarkably provides a method for the use of reverse thermosensitive polymer compositions for the rapid, simple and definitive closure of punctured arteries after peripheral arterial catheterization procedures, without the need for time-consuming manual compression, without the complexity of mechanical devices, and without the risks of embolization associated with collagen plugs.

### SUMMARY OF THE INVENTION

One aspect of the present invention relates to a method to control biological fluid flow at a site in a mammal by use of an in situ formed polymer plug. In certain embodiments, the present invention relates to a method to control bleeding following a catheterization procedure, a method to control leakage of cerebral spinal fluid following a lumbar puncture, a method to seal a fistula, or a method to control the flow of serous fluid after a lymphadenectomy. In certain embodiments, the polymer plug is generated in situ by temperature changes, pH changes or ionic interactions. In certain embodiments, the polymer plug comprises at least one optionally purified reverse thermosensitive polymer.

### BRIEF DESCRIPTION OF THE FIGURES

FIG. 1 depicts a graph of viscosity as a function of temperature for various solutions of purified poloxamer 407.

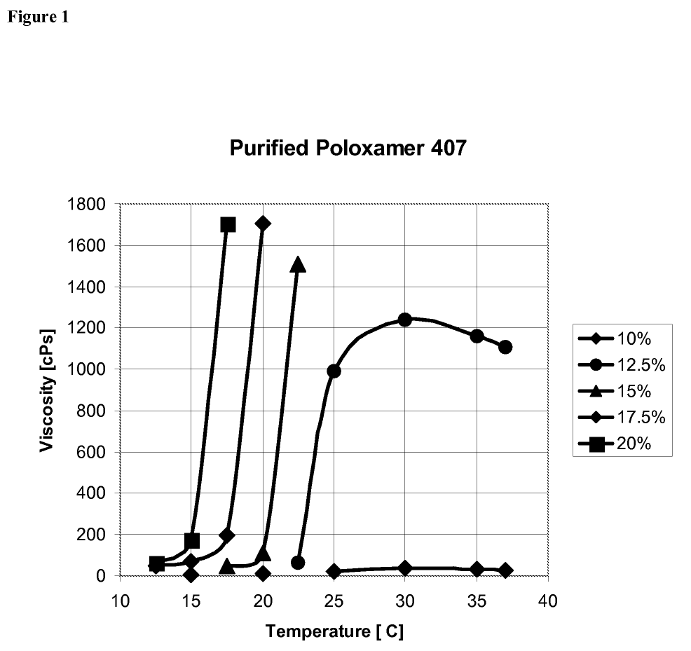

FIG. 2 depicts a table (Table 1) showing the purification of poloxamer 407; and a table (Table 2) showing the gelation temperature of selected reverse thermosensitive polymers in saline. In Table 1, a “*” indicates a viscosity of a 25% solution measured at 30° C. using a cone and plate viscometer.

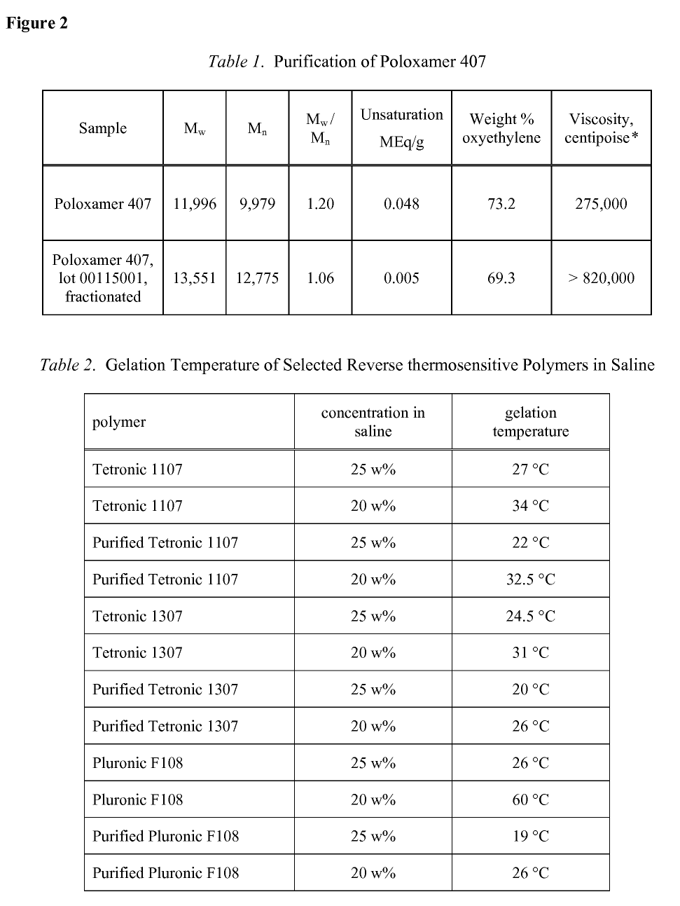

### DETAILED DESCRIPTION OF THE INVENTION

Remarkably, a method and formulation to occlude punctured arteries after peripheral arterial catheterization has been discovered, comprising, in certain embodiments, the following steps: (1) the catheter introducer is removed; (2) a reverse thermosensitive polymer solution or gel is injected directly into the puncture wound; (3) the reverse thermosensitive solution or gel increases in viscosity at body temperature to form a plug; (4) the plug persists long enough to allow for natural hemostasis to occur.

This method eliminates the potential complications associated with gelatin plugs (described above), because the polymer composition is water soluble and non-thrombogenic; therefore, any polymer that penetrates the artery will rapidly dissolve in flowing blood. Additionally, the low viscosity of the reverse thermosensitive polymer solution at room temperature enables its injection into the puncture wound without the need to use an introducer.

Moreover, the invention has been reduced to practice in pigs. Specifically, the introduction of a reverse thermosensitive polymer solution was observed to create rapid hemostasis of the femoral artery and carotid artery access sites while maintaining a patent artery. In all experiments described herein, hemostasis of the access site was achieved within 50 seconds of post-deployment compression. In some of the experiments, hemostasis was observed immediately after the first compression, with compression lasting only 20 seconds in 3 experiments and 40-45 seconds in the other two. Hemostasis continued in all cases until termination of the experiment to enable an exploratory cut down or until animal sacrifice. The longest duration observed was 90 minutes. In some of the experiments, the vessel was observed to be patent immediately post-deployment of the reverse thermosensitive polymer solution. In certain cases, a temporary occlusion of the vessel occurred, followed by a full re-opening of the vessel after 40 minutes in one case and a partial reopening of the vessel after 30 minutes in another case. In this latter case, the experiment was terminated and the animal sacrificed prior to full reopening due to time constraints. In one experiment, the vessel was fully thrombosed, most likely due to the trauma incurred by the vessel while locating the arteriotomy. It is worth noting that that these were not “clean” sticks. They required multiple attempts to gain access to the femoral artery which may have caused damage. The thrombosed vessel revealed by cut-down may be the result of clots caused by failed attempts.

Importantly, while it is important to maintain a patent artery in order to allow the natural healing of the arteriotomy, there is not a safety concern directly associated with reverse thermosensitive polymer solution entering the vessel. The polymer that comprises the reverse thermosensitive polymer solutions have been shown to be biocompatible and non-toxic. Such solutions have been used in temporary vascular occlusion devices and have been shown to dissolve in time after temporarily plugging a vessel to achieve the desired viscous polymer composition occlusion. Once dissolved, the reverse thermosensitive polymer cannot re-solidify, thus alleviating any potential concerns about distal embolism.

In addition to methods for the closure of punctured arteries after peripheral arterial catheterization procedures. the methods described herein can also be used to solve problems related to controlling the flow of biological fluids, for example, in lumbar punctures, treating unwanted fistulas, and lymphadenctomies.

A lumbar puncture, also known as a spinal tap, is performed to withdraw cerebrospinal fluid (CSF), but may result in post-procedure leakage of CSF for days. The state of the art solution employs a blood clot made from the patient's blood to seal the channel. Unfortunately, a patient's clot provides a material with unpredictable quality, such as variable viscosity and sterility. Also, removing the patients blood is cumbersome and time consuming. Remarkably, the present invention solves this problem by utilizing a sterile, ready-to-use reverse thermosensitive polymer composition with known viscosity parameters.

Moreover, unwanted fistulas can be sealed using a viscous material to prevent the flow of bodily fluid from one area to another, such as anal fistulas. In medicine, a fistula is an abnormal connection or passageway between two epithelium-lined organs or vessels that normally do not connect. Remarkably, the present invention solves this problem by utilizing a sterile, ready-to-use reverse thermosensitive polymer composition with known viscosity parameters. The viscous material temporarily occupies space and prevents the flow of fluid from one area to another.

Lymphadenectomy (lymph node removal) typically results in lymph flowing into the area from which a node has been removed and oftentimes results in a seroma. A seroma is a pocket of clear serous fluid that sometimes develops in the body after surgery. A viscous material can be used to occupy temporarily space, thus preventing a seroma. Remarkably, the present invention solves this problem by utilizing a sterile, ready-to-use reverse thermosensitive polymer composition with known viscosity parameters.

### SELECTED ADVANTAGES OF THE INVENTION

Importantly, the inventive compositions and methods have distinct advantages over the materials and methods currently on the market. The invention makes it possible to occlude effectively a puncture site, fisulas or voids created by a lymphadenctomy, while reducing any risk of, for example, arterial embolization or seroma. A delivery system may be used to facilitate and control injection of the reverse thermosensitive polymer composition.

The polymer plugs of the invention can be formed from reverse thermosensitive polymers or other viscous polymer compositions, as long as long as these compositions undergo a physical or chemical transformation when delivered into the puncture site, allowing them to form a plug. Preferably, the composition is easily soluble in flowing blood to minimize the risk of embolization.

### DEFINITIONS

For convenience, before further description of the present invention, certain terms employed in the specification, examples, and appended claims are collected here. These definitions should be read in light of the remainder of the disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time.

The term “poloxamer” denotes a symmetrical block copolymer, consisting of a core of PPG polyoxyethylated to both its terminal hydroxyl groups, i.e., conforming to the interchangable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “poloxamine” denotes a polyalkoxylated symmetrical block copolymer of ethylene diamine conforming to the general type [(PEG)_{X}-(PPG)_{Y}]₂—NCH₂CH₂N—[(PPG)_{Y}—(PEG)_{X}]₂. Each Poloxamine name is followed by an arbitrary code number, which is related to the average numerical values of the respective monomer units denoted by X and Y.

The term “reverse thermosensitive polymer” as used herein refers to a polymer that is soluble in water at ambient temperature, but at least partially phase-separates out of water at physiological temperature. Reverse thermosensitive polymers include, for example, poloxamer 407, poloxamer 188, Pluronic® F127, Pluronic® F68, poly(N-isopropylacrylamide), poly(methyl vinyl ether), poly(N-vinylcaprolactam); and certain poly(organophosphazenes). See: B. H. Lee, et al. “Synthesis and Characterization of Thermosensitive Poly(organophosphazenes) with Methoxy-Poly(ethylene glycol) and Alkylamines as Side Groups,” Bull. Korean Chem. Soc. 2002, 23, 549-554.

The terms “reversibly gelling” and “reverse thermosensitive” refer to the property of a polymer wherein gelation takes place upon an increase in temperature, rather than a decrease in temperature.

The term “transition temperature” refers to the temperature or temperature range at which gelation of an reverse thermosensitive polymer occurs.

The term “degradable”, as used herein, refers to having the property of breaking down or degrading under certain conditions, e.g., by dissolution.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

The term “biocompatible”, as used herein, refers to having the property of being biologically compatible by not producing a toxic, injurious, or immunological response in living tissue.

As used herein “cold-packs” are two containers containing chemicals separated by a frangible seal. When the seal is broken, as the contents from the separate containers begin to react, energy is absorbed from the surroundings creating a cooling effect. An example of chemicals which can be mixed in a cold pack are ammonium nitrate and water. In certain embodiments the cold pack has two sealed bags, one inside the other. The outer bag is made of thick strong plastic. It contains a ammonium nitrate and the second plastic bag. The second (inner) bag is made of a thin weak plastic and contains water. When the bag is squeezed the inner bag breaks and the water mixes with the powder creating the cooling effect.

The term “hemostasis” refers to the stoppage of blood flow through a blood vessel or organ of the body. Hemostasis generally refers to the arrest of bleeding, whether it be by normal vasoconstriction (the vessel walls closing temporarily), by an abnormal obstruction (such as a plaque) or by coagulation or surgical means (such as ligation). As used herein, hemostasis is achieved by using a viscous polymer solution to create an obstruction.

Contemplated equivalents of the polymers, subunits and other compositions described above include such materials which otherwise correspond thereto, and which have the same general properties thereof (e.g., biocompatible), wherein one or more simple variations of substituents are made which do not adversely affect the efficacy of such molecule to achieve its intended purpose. In general, the compounds of the present invention may be prepared by, for example, described below, or by modifications thereof, using readily available starting materials, reagents and conventional synthesis procedures. In these reactions, it is also possible to make use of variants which are in themselves known, but are not mentioned here.

### Reverse Thermosensitive Polymers

In certain embodiments, the methods of the invention may be accomplished by the use of polymers that form a plug inside the body and then dissolve or are dissolved, such as other reverse thermosensitive polymers and any polymer solution or combination of polymers that form a gel inside the body, being under the effect of temperature, pH, pressure, or as a result of a chemical or biological reaction. In other embodiment, the viscous polymer solutions used in a method of the invention are crosslinkable polymers. In certain embodiments, the viscous polymer solutions can be generated in situ. In certain embodiments, the viscous polymer solutions can be non-tissue adhesive.

In certain embodiments, two solutions, a polymer solution and a crosslinker solution, are injected separately (e.g., through a dual lumen catheter) into a biological lumen wherein they gel, forming a viscous polymer solution. The polymer solution may comprise an anionic polymer, a cationic polymer or a non-ionically crosslinkable polymer. Such polymers may comprise one or more of the following: alginic acid, sodium alginate, potassium alginate, sodium gellan, potassium gellan, carboxy methyl cellulose, hyaluronic acid, and polyvinyl alcohol. The cross-linking of the polymer to form a polymer gel may be achieved with anionic crosslinking ions, cationic crosslinking ions, or non-ionic crosslinking agents. Crosslinking agents include, but are not limited to, one or more of the following: phosphate, citrate, borate, succinate, maleate, adipate, oxalate, calcium, magnesium, barium and strontium. Exemplary pairings of polymers and crosslinkers include anionic polymer monomers with cations, such as, for example, alginates with calcium, barium or magnesium; gellans with calcium, magnesium or barium; or hyaluronic acid with calcium. An example of an exemplary pairing of a non-ionic polymer with a chemical crosslinking agent is a polyvinyl alcohol with borate (at a slightly alkaline pH).

In general, the polymers used in the methods of the invention, which become a gel at or about body temperature, can be administered in a liquid form. In certain embodiments, the polymer composition of the invention may be a flexible or flowable material. By “flowable” is meant the ability to assume, over time, the shape of the space containing it at body temperature. This characteristic includes, for example, liquid compositions that are suitable for: injection with a manually operated syringe fitted with, for example, a needle; or delivery through a catheter. Also encompassed by the term “flowable” are highly viscous, gel-like materials at room temperature that may be delivered to the desired site by pouring, squeezing from a tube, or being injected with any one of the commercially available power injection devices that provide injection pressures greater than would be exerted by manual means alone. When the polymer used is itself flowable, the polymer composition of the invention, even when viscous, need not include a biocompatible solvent to be flowable, although trace or residual amounts of biocompatible solvents may be present.

In addition, in certain embodiments, the viscous polymer solution of the invention may be aqueous solution of one or more reverse thermosensitive polymers. These polymer solutions are liquids below body temperature and gel at about body temperature. In certain embodiments, the polymer solution is prepared external of the body, i.e., at a temperature below body temperature. The polymer solution may be further chilled to prolong the time the gel stays in the liquid form upon introduction into the body. A preferred temperature is about 10° C. below the gelation temperature of the polymer solution. In certain embodiments, the viscous polymer solution used in connection with the methods of the invention may comprise a block copolymer with inverse thermal gelation properties. The block copolymer can further comprise a polyoxyethylene-polyoxypropylene block copolymer, such as a biodegradable, biocompatible copolymer of polyethylene oxide and polypropylene oxide. Also, the reverse thermosensitive polymer can include one or more additives; for example, therapeutic agents may be added to the reverse thermosensitive polymers.

In certain embodiments, the block copolymers have molecular weights ranging from about 2,000 to about 1,000,000 Daltons, more particularly at least about 10,000 Daltons, and even more specifically at least about 25,000 Daltons or even at least about 50,000 Daltons. In certain embodiment, the block copolymers have a molecular weight between about 5,000 Daltons and about 30,000 Daltons. In certain embodiments, the molecular weight of the reverse thermosensitive polymer may be between about 1,000 and about 50,000 Daltons, or between about 5,000 and about 35,000 Daltons. In other embodiments, the molecular weight of a suitable reverse thermosensitive polymer (such as a poloxamer or poloxamine) may be, for example, between about 5,000 and about 25,000 Daltons, or between about 7,000 and about 20,000 Daltons. Number-average molecular weight (Mₙ) may also vary, but will generally fall in the range of about 1,000 to about 400,000 Daltons, in some embodiments from about 1,000 to about 100,000 Daltons and, in other embodiments, from about 1,000 to about 70,000 Daltons. In certain embodiments, Mₙ varies between about 5,000 and about 300,000 Daltons.

In certain embodiments, the polymer is in an aqueous solution. For example, typical aqueous solutions contain about 5% to about 30% polymer, preferably about 10% to about 25%. The pH of the reverse thermosensitive polymer formulation administered to a mammal is, generally, about 6.0 to about 7.8, which are suitable pH levels for injection into the mammalian body. The pH level may be adjusted by any suitable acid or base, such as hydrochloric acid or sodium hydroxide.

In certain embodiments, the reverse thermosensitive polymers of the invention are poloxamers or poloxamines. Pluronic® polymers have unique surfactant abilities and extremely low toxicity and immunogenic responses. These products have low acute oral and dermal toxicity and low potential for causing irritation or sensitization, and the general chronic and sub-chronic toxicity is low. In fact, Pluronic® polymers are among a small number of surfactants that have been approved by the FDA for direct use in medical applications and as food additives. See: BASF (1990) Pluronic® & Tetronic® Surfactants, BASF Co., Mount Olive, N.J. Recently, several Pluronic® polymers have been found to enhance the therapeutic effect of drugs, and the gene transfer efficiency mediated by adenovirus. K. L. March, J. E. Madison, and B. C. Trapnell, “Pharmacokinetics of adenoviral vector-mediated gene delivery to vascular smooth muscle cells: modulation by poloxamer 407 and implication for cardiovascular gene therapy,” Hum Gene Therapy 1995, 6, 41-53.

Interestingly, poloxamers (or Pluronics), as nonionic surfactants, are widely used in diverse industrial applications. See, for example, Nonionic Surfactants: polyoxyalkylene block copolymers, Vol. 60. Nace V M, Dekker M (editors), New York, 1996. 280 pp. Their surfactant properties have been useful in detergency, dispersion, stabilization, foaming, and emulsification. A. Cabana, A. K. Abdellatif, and J. Juhasz, “Study of the gelation process of polyethylene oxide. polypropylene oxide-polyethylene oxide copolymer (poloxamer 407) aqueous solutions.” Journal of Colloid and Interface Science 1997, 190, 307-312. Certain poloxamines, e.g., poloxamine 1307 and 1107, also display inverse thermosensitivity.

Importantly, several members of this class of polymer, poloxamer 188, poloxamer 407, poloxamer 338, poloxamine 1107 and poloxamine 1307 show inverse thermosensitivity within the physiological temperature range. Y. Qiu, and K. Park, “Environment-sensitive hydrogels for drug delivery.” Adv Drug Deliv Rev 2001, 53(3), 321-339; and E. S. Ron, and L. E. Bromberg, “Temperature-responsive gels and thermogelling polymer matrices for protein and peptide delivery,” Adv Drug Deliv Rev 1998, 31(3), 197-221. In other words, these polymers are members of a class that are soluble in aqueous solutions at low temperature, but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. As noted above, certain poly(ethyleneoxide)/poly(propyleneoxide) block copolymers have these properties; they are available commercially as Pluronic® poloxamers and Tetronic® poloxamines (BASF, Ludwigshafen, Germany) and generically known as poloxamers and poloxamines, respectively. See U.S. Pat. Nos. 4,188,373, 4,478,822 and 4,474,751; all of which are hereby incorporated by reference.

The average molecular weights of commercially available poloxamers and poloxamines range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15% to about 50% of the total mass of the commercial polymer.

The reverse thermosensitive polymers may be purified using a process for the fractionation of water-soluble polymers, comprising the steps of dissolving a known amount of the polymer in water, adding a soluble extraction salt to the polymer solution, maintaining the solution at a constant optimal temperature for a period of time adequate for two distinct phases to appear, and separating physically the phases. Additionally, the phase containing the polymer fraction of the preferred molecular weight may be diluted to the original volume with water, extraction salt may be added to achieve the original concentration, and the separation process repeated as needed until a polymer having a narrower molecular weight distribution than the starting material and optimal physical characteristics can be recovered.

In certain embodiments, a purified poloxamer or poloxamine has a polydispersity index from about 1.5 to about 1.0. In certain embodiments, a purified poloxamer or poloxamine has a polydispersity index from about 1.2 to about 1.0.

The aforementioned process consists of forming an aqueous two-phase system composed of the polymer and an appropriate salt in water. In such a system, a soluble salt can be added to a single phase polymer-water system to induce phase separation to yield a high salt, low polymer bottom phase, and a low salt, high polymer upper phase. Lower molecular weight polymers partition preferentially into the high salt, low polymer phase. Polymers that can be fractionated using this process include polyethers, glycols such as poly(ethylene glycol) and poly(ethylene oxide)s, polyoxyalkylene block copolymers such as poloxamers, poloxamines, and polyoxypropylene/polyoxybutylene copolymers, and other polyols, such as polyvinyl alcohol. The average molecular weight of these polymers may range from about 800 to greater than 100,000 Daltons. See U.S. Pat. No. 6,761,824 (hereby incorporated by reference). The aforementioned purification process inherently exploits the differences in size and polarity, and therefore solubility, among the poloxamer molecules, the poly(oxyethylene) homopolymer and the poly(oxyethylene)/poly(oxypropylene) diblock byproducts. The polar fraction of the poloxamer, which generally includes the lower molecular weight fraction and the byproducts, is removed allowing the higher molecular weight fraction of poloxamer to be recovered. The larger molecular weight poloxamer recovered by this method has physical characteristics substantially different from the starting material or commercially available poloxamer including a higher average molecular weight, lower polydispersity and a higher viscosity in aqueous solution.

Other purification methods may be used to achieve the desired outcome. For example, WO 92/16484 (hereby incorporated by reference) discloses the use of gel permeation chromatography to isolate a fraction of poloxamer 188 that exhibits beneficial biological effects, without causing potentially deleterious side effects. The copolymer thus obtained had a polydispersity index of 1.07 or less, and was substantially saturated. The potentially harmful side effects were shown to be associated with the low molecular weight, unsaturated portion of the polymer, while the medically beneficial effects resided in the uniform higher molecular weight material. Other similarly improved copolymers were obtained by purifying either the polyoxypropylene center block during synthesis of the copolymer, or the copolymer product itself (e.g., U.S. Pat. No. 5,523,492 and U.S. Pat. No. 5,696,298; both of which are hereby incorporated by reference).

Further, a supercritical fluid extraction technique has been used to fractionate a polyoxyalkylene block copolymer as disclosed in U.S. Pat. No. 5,567,859 (hereby incorporated by reference). A purified fraction was obtained, which was composed of a fairly uniform polyoxyalkylene block copolymer having a polydispersity of less than 1.17. According to this method, the lower molecular weight fraction was removed in a stream of carbon dioxide maintained at a pressure of 2200 pounds per square inch (psi) and a temperature of 40° C.

Additionally, U.S. Pat. No. 5,800,711 (hereby incorporated by reference) discloses a process for the fractionation of polyoxyalkylene block copolymers by the batchwise removal of low molecular weight species using a salt extraction and liquid phase separation technique. Poloxamer 407 and poloxamer 188 were fractionated by this method. In each case, a copolymer fraction was obtained which had a higher average molecular weight and a lower polydispersity index as compared to the starting material. However, the changes in polydispersity index were modest and analysis by gel permeation chromatography indicated that some low-molecular-weight material remained. The viscosity of aqueous solutions of the fractionated polymers was significantly greater than the viscosity of the commercially available polymers at temperatures between 10° C. and 37° C., an important property for some medical and drug delivery applications. Nevertheless, some of the low molecular weight contaminants of these polymers are thought to cause deleterious side effects when used inside the body, making it especially important that they be removed in the fractionation process. As a consequence, polyoxyalkylene block copolymers fractionated by this process are not appropriate for all medical uses.

Modification of the transition temperature of a reverse thermosensitive polymer can be obtained in a number of ways. For example, the transition temperature can be modified either through the addition of transition temperature modifying additive or through the development of a modified polymer. The transition temperature can be influenced by a number of additives, e.g., the addition of pharmaceutical fatty acid excipients such as sodium oleate, sodium laurate or sodium caprate. Other possible pharmaceutical excipients may be solvents such as water, alcohols, especially C₁-C₅ alcohols such as ethanol, n-propanol, 2-propanol, isopropanol, t-butyl alcohol; ethers such as MTBE; ketones such as acetone, methyl ethyl ketone; humectants such as glycerol; glycols such as ethylene glycol, propylene glycol; emulsifiers such as lower, optionally polyhydric C₁-C₅ alcohols partially esterified with long-chain (C₁₂-C₂₄) fatty acids such as glycerol monostearate, isopropyl myristate, fatty acid ester of sugar alcohols such as sorbitan mono-fatty acid ester, polyethoxylated derivatives of such compounds, polyethoxyethylene fatty acid ester and fatty alcohol ether, cholesterol, cetyl stearyl alcohol, wool wax alcohols and synthetic surfactants with a low HLB value; solubilisers such as carbopol; low-viscosity paraffins, triglycerides; lipophilic substances such as isopropyl myristate; pH regulators such as TEA, carbonates and phosphates; chelating agents such as EDTA and salts thereof, as well as preservatives. Furthermore, the addition of other poloxamers to form mixtures of poloxamers is known to influence the transition temperature.

In certain embodiments, to aid in visualization, a contrast-enhancing agent can be added to the viscous polymer compositions of the invention. Exemplarily contrast-enhancing agents are radiopaque materials, paramagnetic materials, heavy atoms, transition metals, lanthanides, actinides, dyes, and radionuclide-containing materials.

### Selected Therapeutic Agents

The reversibly gelling polymers used in the methods of the invention have physico-chemical characteristics that make them suitable delivery vehicles for conventional small-molecule drugs, as well as macromolecular (e.g., peptides) drugs or other therapeutic products. Therefore, the composition comprising the thermosensitive polymer may further comprise a pharmaceutic agent selected to provide a pre-selected pharmaceutic effect. A pharmaceutic effect is one which seeks to prevent or treat the source or symptom of a disease or physical disorder. Pharmaceutics include those products subject to regulation under the FDA pharmaceutic guidelines. Importantly, the compositions used in methods of the invention are capable of solubilizing and releasing bioactive materials. Solubilization is expected to occur as a result of dissolution in the bulk aqueous phase or by incorporation of the solute in micelles created by the hydrophobic domains of the poloxamer. Release of the drug would occur through diffusion or network erosion mechanisms.

Those skilled in the art will appreciate that the compositions used in the methods of the invention may simultaneously be utilized to deliver a wide variety of pharmaceutics to a wound site. To prepare a pharmaceutic composition, an effective amount of pharmaceutically active agent(s), which imparts the desirable pharmaceutic effect is incorporated into the reversibly gelling composition used in the methods of the invention. Preferably, the selected agent is water soluble, which will readily lend itself to a homogeneous dispersion throughout the reversibly gelling composition. It is also preferred that the agent(s) is non-reactive with the composition. For materials, which are not water soluble, it is also within the scope of the methods of the invention to disperse or suspend lipophilic material throughout the composition. Myriad bioactive materials may be delivered using the methods of the present invention; the delivered bioactive material includes anesthetics, antimicrobial agents (antibacterial, antifungal, antiviral), anti-inflammatory agents, diagnostic agents, and wound-healing agents.

Because the reversibly gelling composition used in the methods of the present invention are suited for application under a variety of environmental conditions, a wide variety of pharmaceutically active agents may be incorporated into and administered via the composition. The pharmaceutic agent loaded into the polymer networks of the thermosensitive polymer may be any substance having biological activity, including proteins, polypeptides, polynucleotides, nucleoproteins, polysaccharides, glycoproteins, lipoproteins, and synthetic and biologically engineered analogs thereof.

A vast number of therapeutic agents may be incorporated in the polymers used in the methods of the present invention. In general, therapeutic agents which may be administered via the methods of the invention include, without limitation: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins. Suitable pharmaceuticals for parenteral administration are well known as is exemplified by the Handbook on Injectable Drugs, 6th Edition, by Lawrence A. Trissel, American Society of Hospital Pharmacists, Bethesda, Md., 1990 (hereby incorporated by reference).

The pharmaceutically active compound may be any substance having biological activity, including proteins, polypeptides, polynucleotides, nucleoproteins, polysaccharides, glycoproteins, lipoproteins, and synthetic and biologically engineered analogs thereof. The term “protein” is art-recognized and for purposes of this invention also encompasses peptides. The proteins or peptides may be any biologically active protein or peptide, naturally occurring or synthetic.

Examples of proteins include antibodies, enzymes, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The pharmaceutical agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelop glycoproteins and cytokins and other agents selected from the group consisting of bovine somatropine (sometimes referred to as BST), estrogens, androgens, insulin growth factors (sometimes referred to as IGF), interleukin I, interleukin II and cytokins. Three such cytokins are interferon-β, interferon-γ and tuftsin.

Examples of bacterial toxoids that may be incorporated in the compositions used in the methods of the invention are tetanus, diphtheria, pseudomonas A, mycobaeterium tuberculosis. Examples of that may be incorporated in the compositions used in the occlusion methods of the invention are HIV envelope glycoproteins, e.g., gp120 or gp 160, for AIDS vaccines. Examples of anti-ulcer H2 receptor antagonists that may be included are ranitidine, cimetidine and famotidine, and other anti-ulcer drugs are omparazide, cesupride and misoprostol. An example of a hypoglycaemic agent is glizipide.

Classes of pharmaceutically active compounds which can be loaded into that may be incorporated in the compositions used in the occlusion methods of the invention include, but are not limited to, anti-AIDS substances, anti-cancer substances, antibiotics, immunosuppressants (e.g., cyclosporine) anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NTHEs, local anesthetics, ophthalmics, prostaglandins, anti-depressants, anti-psychotic substances, anti-emetics, imaging agents, specific targeting agents, neurotransmitters, proteins, cell response modifiers, and vaccines.

Exemplary pharmaceutical agents considered to be particularly suitable for incorporation in the compositions used in the methods of the invention include but are not limited to imidazoles, such as miconazole, econazole, terconazole, saperconazole, itraconazole, metronidazole, fluconazole, ketoconazole, and clotrimazole, luteinizing-hormone-releasing hormone (LHRH) and its analogues, nonoxynol-9, a GnRH agonist or antagonist, natural or synthetic progestrin, such as selected progesterone, 17-hydroxyprogeterone derivatives such as medroxyprogesterone acetate, and 19-nortestosterone analogues such as norethindrone, natural or synthetic estrogens, conjugated estrogens, estradiol, estropipate, and ethinyl estradiol, bisphosphonates including etidronate, alendronate, tiludronate, resedronate, clodronate, and pamidronate, calcitonin, parathyroid hormones, carbonic anhydrase inhibitor such as felbamate and dorzolamide, a mast cell stabilizer such as xesterbergsterol-A, Iodoxamine, and cromolyn, a prostaglandin inhibitor such as diclofenac and ketorolac, a steroid such as prednisolone, dexamethasone, fluoromethylone, rimexolone, and lotepednol, an antihistamine such as antazoline, pheniramine, and histiminase, pilocarpine nitrate, a beta-blocker such as levobunolol and timolol maleate. As will be understood by those skilled in the art, two or more pharmaceutical agents may be combined for specific effects. The necessary amounts of active ingredient can be determined by simple experimentation.

By way of example only, any of a number of antibiotics and antimicrobials may be included in the thermosensitive polymers used in the methods of the invention. Antimicrobial drugs preferred for inclusion in compositions used in the occlusion methods of the invention include salts of lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole and amanfadine and the like.

By way of example only, in the case of anti-inflammation, non-steroidal anti-inflammatory agents (NTHES) may be incorporated in the compositions used in the occlusion methods of the invention, such as propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid and the like.

### Injection Systems

A delivery system may be used to facilitate and control injection of the reverse thermosensitive polymer composition. Ideally, the injection system would minimize the need for dissection of the artery prior to injection. Further, in constructing an optimal injection system it may be helpful to determine the thumb pressure required to inject the polymer in liquid form through various diameter needles while maintaining a flow rate of 0.5 mL per second. A tensile testing apparatus (e.g., Instron®) can be used measure the force needed and resulting rate of compression to depress the plunger.

In certain embodiments, use of a cannula that can be detected in a vessel using standard non-invasive systems in the operating room (e.g., a handheld ultrasound) will aid in verifying that the cannula is correctly placed in the renal artery. The catheter may be a dilatation catheter. In one embodiment, the catheter is 3-10 French in size, and more preferably 3-6 French. In another embodiment, a catheter can be used to dispense one or more fluids other than, or in addition to, the polymer solution. In the embodiment the catheter may be a multiple lumen catheter with one lumen for the delivery of the polymer solution, other lumen for the delivery of other fluids such as a contrast agent solution.

In another embodiment, the syringe or other mechanism may be used to inject the polymer solution into the body can be, for example, a 1-100 cc syringe, a 1-50 cc syringe or a 1-5 cc. Pressure applied to the syringe can be applied by hand or by an automated syringe pusher. In certain embodiments, a system to provide auxiliary power to a syringe for injection of a viscous material (e.g., a spring loaded plunger assisted device) may be used.

### METHODS OF THE INVENTION

One aspect of the present invention relates to a method to control biological fluid flow at a site in a mammal by use of an in situ formed polymer plug, comprising the step of:

allowing a viscous polymer composition to solidify at body temperature, thereby forming the polymer plug in situ.

In certain embodiments, the present invention relates to any of the aforementioned methods and any of the attendant limitations, further comprising the step of injecting a viscous polymer composition directly into the site.

In certain embodiments the present invention relates to any one of the aforementioned methods and any of the attendant limitations, wherein the polymer plug is generated in situ by temperature changes, pH changes or ionic interactions.

In certain embodiments, the present invention relates to any one of the aforementioned methods and any of the attendant limitations, further comprising the steps of injecting a first composition directly into the site in a mammal; and injecting a second composition directly into the site in a mammal, wherein the first composition contacts the second composition, thereby forming the viscous polymer composition in situ.

In certain embodiments, the present invention relates to any one of the aforementioned methods and any of the attendant limitations, wherein the first composition and the second composition are injected separately.

In certain embodiments, the present invention relates to any one of the aforementioned methods and any of the attendant limitations, wherein the first composition and the second composition are injected simultaneously.

In certain embodiments, the present invention relates to any one of the aforementioned methods and any of the attendant limitations, wherein the method controls bleeding following a catheterization procedure, controls leakage of cerebral spinal fluid following a lumbar puncture, seals a fistula, or controls the flow of serous fluid after a lymphadenectomy.

In certain embodiments, the present invention relates to any one of the aforementioned methods and any of the attendant limitations, wherein the method controls bleeding following a catheterization procedure; and the site is a puncture of a lumen resulting from the catheterization.

In certain embodiments, the present invention relates to any one of the aforementioned methods and any of the attendant limitations, wherein the method control leakage of cerebral spinal fluid following a lumbar puncture; and the site is a puncture of a lumen resulting from the lumbar puncture.

In certain embodiments, the present invention relates to any one of the aforementioned methods and any of the attendant limitations, wherein the method seal a fistula; and the site is an abnormal connection or passageway between two epithelium-lined organs or vessels that normally do not connect.

In certain embodiments, the present invention relates to any one of the aforementioned methods and any of the attendant limitations, wherein the method control the flow of serous fluid after a lymphadenectomy; and the site is an void resulting from the lymphandenctomy.

In certain embodiments, the present invention relates to any one of the aforementioned methods and any of the attendant limitations, wherein the volume of the viscous polymer composition is about 1-25 mL.

In certain embodiments, the present invention relates to any one of the aforementioned methods and any of the attendant limitations, wherein the volume of the viscous polymer composition is about 1-10 mL.

In certain embodiments, the present invention relates to any one of the aforementioned methods and any of the attendant limitations, wherein the viscous polymer composition is introduced over about 30 seconds.

In certain embodiments, the present invention relates to any one of the aforementioned methods and any of the attendant limitations, wherein the viscous polymer composition is introduced over about 20 seconds.

In certain embodiments, the present invention relates to any one of the aforementioned methods and any of the attendant limitations, wherein the viscous polymer composition is introduced over about 10 seconds.

In certain embodiments, the present invention relates to any one of the aforementioned methods and any of the attendant limitations, wherein the viscous polymer composition is a solid at mammalian physiological temperature.

In certain embodiments, the present invention relates to any one of the aforementioned methods and any of the attendant limitations, wherein the viscous polymer composition comprises at least one optionally purified reverse thermosensitive polymer.

In certain embodiments, the present invention relates to any one of the aforementioned methods and any of the attendant limitations, wherein the viscous polymer composition comprises about 5% to about 35% of the reverse thermosensitive polymer.

In certain embodiments, the present invention relates to any one of the aforementioned methods and any of the attendant limitations, wherein the viscous polymer composition comprises about 10% to about 30% of the reverse thermosensitive polymer.

In certain embodiments, the present invention relates to any one of the aforementioned methods and any of the attendant limitations, wherein the viscous polymer composition comprises about 20% of the reverse thermosensitive polymer.

In certain embodiments, the present invention relates to any one of the aforementioned methods and any of the attendant limitations, wherein the at least one optionally purified reverse thermosensitive polymer has a polydispersity index from about 1.5 to about 1.0.

In certain embodiments, the present invention relates to any one of the aforementioned methods and any of the attendant limitations, wherein the at least one optionally purified reverse thermosensitive polymer has a polydispersity index from about 1.2 to about 1.0.

In certain embodiments, the present invention relates to any one of the aforementioned methods and any of the attendant limitations, wherein the at least one optionally purified reverse thermosensitive polymer is selected from the group consisting of block copolymers, random copolymers, graft polymers, and branched copolymers.

In certain embodiments, the present invention relates to any one of the aforementioned methods and any of the attendant limitations, wherein the at least one optionally purified reverse thermosensitive polymer is a polyoxyalkylene block copolymer.

In certain embodiments, the present invention relates to any one of the aforementioned methods and any of the attendant limitations, wherein the at least one optionally purified reverse thermosensitive polymer is selected from the group consisting of poloxamers and poloxamines.

In certain embodiments, the present invention relates to any one of the aforementioned methods and any of the attendant limitations, wherein the at least one optionally purified reverse thermosensitive polymer is selected from the group consisting of poloxamer 407, poloxamer 288, poloxamer 188, poloxamer 338, poloxamer 118, Tetronic® 1107 and Tetronic® 1307.

In certain embodiments, the present invention relates to any one of the aforementioned methods and any of the attendant limitations, wherein the at least one optionally purified reverse thermosensitive polymer is poloxamer 407.

In certain embodiments, the present invention relates to any one of the aforementioned methods and any of the attendant limitations, wherein the at least one optionally purified reverse thermosensitive polymer is selected from the group consisting of purified poloxamers and purified poloxamines.

In certain embodiments, the present invention relates to any one of the aforementioned methods and any of the attendant limitations, wherein the at least one optionally purified reverse thermosensitive polymer is selected from the group consisting of purified poloxamer 407, purified poloxamer 288, purified poloxamer 188, purified poloxamer 338, purified poloxamer 118, purified Tetronic® 1107 and purified Tetronic® 1307.

In certain embodiments, the present invention relates to any one of the aforementioned methods and any of the attendant limitations, wherein the at least one optionally purified reverse thermosensitive polymer is purified poloxamer 407.

In certain embodiments, the present invention relates to any one of the aforementioned methods and any of the attendant limitations, wherein the viscous polymer composition gel comprises an excipient.

In certain embodiments, the present invention relates to any one of the aforementioned methods and any of the attendant limitations, wherein the viscous polymer composition gel comprises a pharmaceutical fatty acid excipient.

In certain embodiments, the present invention relates to any one of the aforementioned methods and any of the attendant limitations, wherein the pharmaceutical fatty acid excipient is sodium oleate, sodium laurate or sodium caprate.

In certain embodiments, the present invention relates to any one of the aforementioned methods and any of the attendant limitations, wherein the viscous polymer composition gel comprises a therapeutic agent.

In certain embodiments, the present invention relates to any one of the aforementioned methods and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to any one of the aforementioned methods and any of the attendant limitations, wherein the therapeutic agent is an antibiotic.

In certain embodiments, the present invention relates to any of the aforementioned methods and any of the attendant limitations, wherein the viscous polymer composition gel comprises a contrast-enhancing agent.

In certain embodiments, the present invention relates to any of the aforementioned methods and any of the attendant limitations, wherein the contrast-enhancing agent is selected from the group consisting of radiopaque materials, paramagnetic materials, heavy atoms, transition metals, lanthanides, actinides, dyes, and radionuclide-containing materials.

In certain embodiments, the present invention relates to any one of the aforementioned methods and any of the attendant limitations, wherein the viscous polymer composition has a transition temperature of between about 20° C. and about 50° C.

In certain embodiments, the present invention relates to any one of the aforementioned methods and any of the attendant limitations, wherein the viscous polymer composition has a transition temperature of between about 30° C. and about 40° C.

In certain embodiments, the present invention relates to any one of the aforementioned methods and any of the attendant limitations, wherein the volume of the viscous polymer composition at physiological temperature is about 80% to about 120% of its volume below its transition temperature.

In certain embodiments, the present invention relates to any one of the aforementioned methods and any of the attendant limitations, wherein the volume of the viscous polymer composition at physiological temperature is about 80% to about 120% of its volume below its transition temperature; and the viscous polymer composition has a transition temperature of between about 20° C. and about 50° C.

In certain embodiments, the present invention relates to any one of the aforementioned methods and any of the attendant limitations, wherein the volume of the viscous polymer composition at physiological temperature is about 80% to about 120% of its volume below its transition temperature; and the viscous polymer composition has a transition temperature of between about 30° C. and about 40° C.

In certain embodiments, the present invention relates to any one of the aforementioned methods and any of the attendant limitations, wherein the volume of the viscous polymer composition at physiological temperature is about 80% to about 120% of its volume below its transition temperature; the viscous polymer composition has a transition temperature of between about 20° C. and about 50° C.; and the viscous polymer composition comprises at least one optionally purified reverse thermosensitive polymer selected from the group consisting of poloxamers and poloxamines.

In certain embodiments, the present invention relates to any one of the aforementioned methods and any of the attendant limitations, wherein the volume of the viscous polymer composition at physiological temperature is about 80% to about 120% of its volume below its transition temperature; the viscous polymer composition has a transition temperature of between about 30° C. and about 40° C.; and the viscous polymer composition comprises at least one optionally purified reverse thermosensitive polymer selected from the group consisting of poloxamers and poloxamines.

In certain embodiments, the present invention relates to any one of the aforementioned methods and any of the attendant limitations, wherein the viscous polymer composition comprises an anionic, cationic, or non-ionically crosslinkable polymer.

In certain embodiments, the present invention relates to any one of the aforementioned methods and any of the attendant limitations, wherein the viscous polymer composition comprises a polymer selected from the group consisting of alginic acid, sodium alginate, potassium alginate, sodium gellan, potassium gellan, carboxy methyl cellulose, hyaluronic acid and polyvinyl alcohol.

In certain embodiments, the present invention relates to any one of the aforementioned methods and any of the attendant limitations, wherein the viscous polymer composition comprises phosphate, citrate, borate, succinate, maleate, adipate, oxalate, calcium, magnesium, barium, or strontium.

In certain embodiments, the present invention relates to any one of the aforementioned methods and any of the attendant limitations, wherein the viscous polymer composition comprises a polymer selected from the group consisting of alginic acid, sodium alginate, potassium alginate, sodium gellan and potassium gellan; and calcium, magnesium or barium.

In certain embodiments, the present invention relates to any one of the aforementioned methods and any of the attendant limitations, wherein the viscous polymer composition comprises a polymer selected from the group consisting of alginic acid, sodium alginate and potassium alginate; and calcium.

In certain embodiments, the present invention relates to any one of the aforementioned methods and any of the attendant limitations, wherein the viscous polymer composition comprises a polymer selected from the group consisting of sodium gellan and potassium gellan; and magnesium.

In certain embodiments, the present invention relates to any one of the aforementioned methods and any of the attendant limitations, wherein the viscous polymer composition comprises hyaluronic acid; and calcium.

In certain embodiments, the present invention relates to any one of the aforementioned methods and any of the attendant limitations, wherein the viscous polymer composition comprises polyvinyl alcohol; and borate.

In certain embodiments, the present invention relates to any one of the aforementioned methods and any of the attendant limitations, wherein the viscous polymer composition comprises a protein selected from the group consisting of collagen, gelatin, elastin, albumin, protamine, fibrin, fibrinogen, keratin, reelin, and caseine.

In certain embodiments, the present invention relates to any one of the aforementioned methods and any of the attendant limitations, wherein the viscous polymer composition comprises hyaluronic acid, or chitosan.

In certain embodiments, the present invention relates to any one of the aforementioned methods and any of the attendant limitations, wherein the viscous polymer composition comprises alginate, pectin, methylcellulose, or carboxymethylcellulose.

In certain embodiments, the present invention relates to any one of the aforementioned methods and any of the attendant limitations, wherein the viscous polymer composition comprises a crosslinkable polymer.

In certain embodiments, the present invention relates to any one of the aforementioned methods and any of the attendant limitations, wherein the lifetime of the viscous polymer composition is about thirty minutes.

In certain embodiments, the present invention relates to any one of the aforementioned methods and any of the attendant limitations, wherein the lifetime of the viscous polymer composition is about forty minutes.

In certain embodiments, the present invention relates to any one of the aforementioned methods and any of the attendant limitations, wherein the mammal is a human.

In certain embodiments, the present invention relates to any one of the aforementioned methods and any of the attendant limitations, wherein the viscous polymer composition, the first composition, or the second composition, is introduced using a syringe, cannula, catheter or percutaneous access device.

In certain embodiments, the present invention relates to any one of the aforementioned methods and any of the attendant limitations, wherein the viscous polymer composition, the first composition, or the second composition, is introduced using a dual lumen catheter or a triple lumen catheter.

In certain embodiments, the present invention relates to any one of the aforementioned methods and any of the attendant limitations, wherein the catheter is 3-10 French or 3-6 French in size.

In certain embodiments, the present invention relates to any one of the aforementioned methods and any of the attendant limitations, wherein the catheter can be used to dispense one or more fluids other than, or in addition to, the polymer solution. For example, the catheter may be a multiple lumen catheter with one lumen for the delivery of the polymer solution, other lumen for the delivery of other fluids such as a contrast agent solution.

In certain embodiments, the present invention relates to any one of the aforementioned methods and any of the attendant limitations, wherein the viscous polymer composition, the first composition, or the second composition, is introduced using a syringe.

In certain embodiments, the present invention relates to any one of the aforementioned methods and any of the attendant limitations, wherein the syringe used to inject the polymer solution into the body can be a 1-100 cc syringe, a 1-50 cc syringe or a 1-5 cc syringe. Pressure applied to the syringe can be applied by hand or by an automated syringe pusher.

In certain embodiments, the present invention relates to any one of the aforementioned methods and any of the attendant limitations, wherein the viscous polymer composition, the first composition, or the second composition, is cooled to about 15° C. prior to introduction.

In certain embodiments, the present invention relates to any one of the aforementioned methods and any of the attendant limitations, wherein the viscous polymer composition, the first composition, or the second composition, is cooled to about 10° C. prior to introduction.

In certain embodiments, the present invention relates to any one of the aforementioned methods and any of the attendant limitations, wherein the viscous polymer composition, the first composition, or the second composition, is cooled to about 5° C. prior to introduction.

In certain embodiments, the present invention relates to any one of the aforementioned methods and any of the attendant limitations, wherein the viscous polymer composition, the first composition, or the second composition, is cooled to about 0° C. prior to introduction.

In certain embodiments, the present invention relates to any one of the aforementioned methods and any of the attendant limitations, wherein the viscous polymer composition, the first composition, or the second composition, is cooled with ice, water, or a cold pack prior to introduction.

In certain embodiments, the present invention relates to any one of the aforementioned methods and any of the attendant limitations, further comprising introducing saline to aid in the dissolution of the polymer plug.

In certain embodiments, the present invention relates to any one of the aforementioned methods and any of the attendant limitations, further comprising the step of cooling the site.

### Kits

This invention also provides kits for conveniently and effectively implementing the methods of this invention. Such kits comprise any of the polymers of the present invention or a combination thereof, and a means for facilitating their use consistent with methods of this invention. Such kits may also included ice, a cold pack, or other means of cooling. Such kits provide a convenient and effective means for assuring that the methods are practiced in an effective manner. The compliance means of such kits includes any means which facilitates practicing a method of this invention. Such compliance means include instructions, packaging, and dispensing means, and combinations thereof. Kit components may be packaged for either manual or partially or wholly automated practice of the foregoing methods. In other embodiments, this invention contemplates a kit including block copolymers of the present invention, and optionally instructions for their use. In certain embodiments, the reverse thermosensitive copolymers of such a kit of the present invention are contained in one or more syringes.

In certain embodiments, the present invention relates to a kit for conveniently and effectively implementing the method of this invention, comprising instructions for use thereof, and a first container comprising a volume of a composition, wherein the composition forms a viscous polymer composition at mammalian physiological temperature. In certain embodiments, the present invention relates to the aforementioned kit and any of the attendant limitations, further comprising a cold pack. In certain embodiments, the present invention relates to the aforementioned kit and any of the attendant limitations, further comprising a syringe or cannula. In certain embodiments, the present invention relates to the aforementioned kit and any of the attendant limitations, wherein the viscous polymer composition comprises at least one optionally purified reverse thermosensitive polymer, such as those described above.

### EXEMPLIFICATION

The invention, having been generally described, may be more readily understood by reference to the following examples, which are included merely for purposes of illustration of certain aspects and embodiments of the present invention, and are not intended to limit the invention in any way. All headings are for the convenience of the reader and should not be used to limit the meaning of the text that follows the heading, unless so specified.

### Example 1

LeGoo™ (poloxamer 407) at 20% aqueous was used to close a femoral arteries of pigs 1-3, each weighing approximately 30 kilograms.

Experiment 1—Left Femoral Artery On Pig 1. An 8 French introducer was removed and pulsating bleeding was observed. The column of blood rose approximately 4 cm off leg. 3 mL of LeGoo™ was injected (room temperature) using the nose of a syringe only. Bleeding stopped immediately and the wound remained closed for 0.75 hours until the animal was sacrificed.

Experiment 2—Right Femoral Artery On Pig 2. An 8 French introducer was removed and pulsating bleeding was observed. Blood welled up in the groin area rapidly (approximately 10 mL in 2 seconds). 3 mL of LeGoo™ was injected (room temperature) using a 16 gauge cannula. Bleeding stopped within seconds and the wound remained closed for 1.5 hours until the animal was sacrificed.

Experiment 3—Left Femoral Artery On Pig 3. A 10 French introducer was removed and pulsating bleeding was observed. Blood welled up in the groin area very rapidly (faster than Pig 2). 6 mL of LeGoo™ was injected (room temperature) using a 16 gauge cannula. Bleeding stopped within seconds and the wound remained closed for 0.5 hours until the animal was sacrificed.

Further experiments, similar to the experiments described above, to look at longer term effect and verify that the closure subsides after the plug has dissolved in the tissue, are described below.

### Example 2

Exploratory Methods. Seven experiments were performed on the femoral and carotid arteries of 2 female swine. Pig 4 weighed 34 kg and Pig 5 weighed 27 kg. The animals were anesthetized with 2-3% of isoflurane with two part of air for one of O₂ (4:2) in accordance with the Montreal Heart Institute animal care committee protocol.

Access to the femoral and carotid arteries was obtained using conventional percutaneous insertion of a 6 French introducer sheath into the arteries on both sides. For introduction, 8 cc of ketamine (100 mg/mL) plus 0.88 cc xylazine (100 mg/mL) were delivered intramuscularly. The left carotid artery was catheterized to visualize the closure site using contrast media under fluoroscopy. The catheter was inserted via carotid artery through a 6 french and advanced down into the iliac artery of the respected side. Two methods of delivering a reverse thermosensitive polymer solution to the arteriotomy site were employed.

Method 1. A 0.018 guide wire was inserted through the introducer sheath to maintain arterial access when the introducer sheath was removed. A “Locator” sheath was introduced over the wire to locate the depth of the arteriotomy. A “Delivery” sheath was then introduced to the depth identified by the locator sheath. The guide wire was then removed before deployment of a reverse thermosensitive polymer solution on top of the arteriotomy site.

Method 2. A 3 cc syringe was connected to a 6 French dilator. The dilator was inserted through the introducer sheath to the distal tip. The introducer sheath was then withdrawn 2-4 mm above the arteriotomy before deployment of a reverse thermosensitive polymer solution via the dilator.

After deployment of a reverse thermosensitive polymer solution the artery was digitally compressed before assessing hemostasis. Contrast media was injected under fluoroscopy via the carotid catheter to assess vessel patency post deployment. Each animals' assess sites were observed along with vessel patency using fluoroscopy for up to 90 minutes post procedure or until animal sacrifice or experiment termination.

At the completion of the study, the animals were euthanized with 5% isoflurane with two part of air for one of O₂ (4:2) plus 10 mL of KCl 2 mEq/mL, 0.7 mEq/kg delivered intravenously in accordance with the Montreal Heart Institute animal care committee protocol.

Experiment 4—Right Femoral Artery On Pig 4. A 0.2 cc solution of poloxamer 407 at 20% aqueous was cooled via refrigeration until approximately 5 minutes before deployment. The “Locator” sheath and “Delivery” sheath method (method #1) was used.

Locating the arteriotomy required 5 minutes of fairly rigorous manipulation. The access track was dilated to approximately 8-10 french to accommodate the “Locator” sheath. The reverse thermosensitive polymer solution was deployed and digital compression was maintained for 40 seconds. Hemostasis was immediately achieved as noted by no bleeding at the access site. No visible hematoma or swelling in the groin was visible despite significant manipulation. Fluoroscopy confirmed a patent vessel post reverse thermosensitive polymer solution deployment, though the artery appeared to be irregularly shaped at the arteriotomy site, perhaps related to the size of the vessel compared to the size of the 6 french sheath. Fluoroscopy images had neither been captured prior to arteriotomy location nor prior to reverse thermosensitive polymer solution deployment, so it was not possible to confirm this hypothesis. This was corrected in later experiments.

At 60 minutes post deployment, hemostasis of the access site continued and fluoroscopy confirmed patency of the artery though the artery remained irregularly shaped. At 90 minutes, hemostasis continued.

Experiment 5—Left Femoral Artery On Pig 4. A 0.2 cc solution of poloxamer 407 at 20% aqueous, with iohexyl contrast agent added, was cooled via refrigeration until approximately 5 minutes before deployment. The “Locator” sheath and “Delivery” sheath method (Method #1) was used.

Location of arteriotomy again required significant manipulation with the “Locator sheath” resulting in approximately 8-10 french track diameter. Fluoroscopy images taken after the locator sheath was inserted but before the reverse thermosensitive polymer solution was deployed revealed no flow distal to the locator sheath. The reverse thermosensitive polymer solution was deployed and digital compression was maintained for 35-40 seconds. Hemostasis of the access site was achieved immediately after compression with no signs of groin swelling or hematoma. Despite the addition of the iohexyl, the reverse thermosensitive polymer solution was not detectable via fluoroscopy. Fluoroscopy revealed no flow through the artery post deployment. After 30 minutes, hemostasis continued, no signs of hematoma were present, and the artery continued to be occluded.

The experiment was then terminated and a cut down was performed to assess the cause of the occlusion. The cut down revealed the resulting polymer plug still intact and located approximately 1 cm above the artery in the track, indicating that the reverse thermosensitive polymer solution was most likely not deployed directly into the artery. The artery was found to be fairly mangled and completely thrombosed. This occlusion was potentially related to arterial spasm though the cause is unknown. The wound was then sutured and the animal was prepared for subsequent experiments.

Experiment 6—Left Carotid Artery On Pig 4. Poloxamer 407 at 20% aqueous was used. In order to avoid trauma to the vessel at the arteriotomy site, the syringe-sheath delivery system (method #2) was used. While less traumatic, this system is also less accurate in delivering the reverse thermosensitive polymer solution to just above the arteriotomy site. The reverse thermosensitive polymer solution was deployed and followed by digital compression for 25 seconds. Hemostasis was not immediately achieved as steady but un-pressurized “track oozing” ensued. Compression was continued for an additional 20 seconds and hemostasis followed. Fluoroscopy immediately after deployment revealed an occluded carotid artery, potentially due to spasm or the presence of the reverse thermosensitive polymer solution in the vessel. After 30 minutes, fluoroscopy revealed a partially patent vessel, and at 40 minutes post deployment, fluoroscopy revealed a fully patent vessel. Hemostasis of the access site continued until animal sacrifice (after the following experiment) after 50 minutes.

Experiment 7—Right Carotid Artery On Pig 4. Poloxamer 407 at 20% aqueous was used. In order achieve more accurate delivery location, the “Locator” sheath and “Delivery” sheath was again utilized. Locating the arteriotomy required significantly less manipulation compared to prior uses of this system. Fluoroscopy revealed a patent carotid artery post location and pre deployment. The reverse thermosensitive polymer solution was deployed and followed by digital compression for 20 seconds. Hemostasis was immediately achieved with no signs of hematoma. Fluoroscopy immediately after deployment revealed a patent carotid artery. At 30 minutes, fluoroscopy confirmed continued patency and hemostasis of the access site until animal was sacrificed.

Experiment 8—Left Femoral Artery On Pig 5. Poloxamer 407 at 20% aqueous was used. The “Locator” sheath and “Delivery” sheath was again utilized.

Injection of contrast via carotid catheter revealed a fully patent femoral artery prior to 6 French introducer sheath placement. A second injection of contrast via carotid catheter after the introducer sheath was placed revealed no flow through the femoral artery distal to the sheath, possibly due to the presence of the introducer sheath and the relatively small diameter of the vessel. A third injection of contrast via carotid catheter after the 6 French introducer sheath was removed (leaving only the 0.18 wire in place) revealed a fully patent femoral artery. Location of the arteriotomy was performed with ease and a fourth injection via carotid catheter was repeated to reveal a fully patent femoral artery (no spasms).

The reverse thermosensitive polymer solution was then deployed. After 20 seconds of compression, hemostasis was immediately achieved at the access site. Minor track oozing continued for approximately 2 seconds. Immediately post deployment, contrast injection via carotid catheter under fluoroscopy revealed a fully patent vessel. Hemostasis at the access site continued for over 70 minutes until animal was sacrificed (after experiments 9 and 10).

Experiment 9—Right Femoral Artery On Pig 5. Poloxamer 407 at 20% aqueous was used. The “Locator” sheath and “Delivery” sheath was again utilized.

Injection of contrast via carotid catheter revealed a fully patent femoral artery prior to 6 French introducer sheath placement. A second injection of contrast via carotid catheter after the introducer sheath was placed revealed no flow through the femoral artery distal to the sheath, possibly due to the presence of the introducer sheath and the relatively small diameter of the vessel. A third injection of contrast via carotid catheter after the 6 French introducer sheath was removed (leaving only the 0.18 wire in place) revealed a fully patent femoral artery. Location of the arteriotomy was performed with ease and a fourth injection via carotid catheter was repeated to reveal a fully patent femoral artery (no spasms).

An initial attempt to deploy a greater volume (0.3 cc) of the reverse thermosensitive polymer solution failed due to a modification made on the “Delivery” sheath system. While compression was held for approximately 2 minutes, an additional “Delivery” sheath was loaded with 0.2 cc of the reverse thermosensitive polymer solution. Contrast was injected via carotid catheter to confirm the femoral artery was still patent even after the compression and time lapse. Bleeding was noted at the site when manual compression was released demonstrating that the compression did not cause hemostasis prior to deployment. The reverse thermosensitive polymer solution was deployed. After 20 seconds of compression, hemostasis was immediately achieved at the access site. Again, minor track oozing continued for approximately 2 seconds. Immediately post deployment, fluoroscopy revealed a patent femoral artery at the arteriotomy site with flow slightly slowed distal to the arteriotomy. This was most likely due to the extended compression after the failed first deployment. Hemostasis at the access site continued for 56 minutes until animal was sacrificed.

Experiment 10—Re-Access Of Left Femoral Artery On Pig 5. Poloxamer 407 at 20% aqueous was used immediately after withdrawal from an ice bath. The syringe-sheath system delivery method (Method #2) was used.

The left femoral artery was re-accessed. In an effort to explore any changes in performance resulting from variations in the temperature (and hence viscosity) of the reverse thermosensitive polymer solution at the time of deployment, the reverse thermosensitive polymer solution was deployed immediately after removal from an ice bath while still in liquid form. This required the use of the syringe-sheath system since the “Locator” sheath and “Delivery” sheath system was not air tight and could not contain a liquid polymer. 1.5 cc of the reverse thermosensitive polymer solution was deployed and compression was held for 20 seconds. Steady bleeding appeared, followed by another 30 seconds of compression. Hemostasis was then obtained. A mild hematoma was present. Fluoroscopy showed the vessel to be occluded. After 30 minutes, fluoroscopy identified a partial reopening of the vessel at which time the animal was sacrificed due to time constraints.

### EQUIVALENTS

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

### INCORPORATION BY REFERENCE

All of the US patents and US patent application Publications cited herein are hereby incorporated by reference.

We claim:

1. A method to control biological fluid flow at a site in a mammal by use of an in situ formed polymer plug, comprising the step of:
allowing a viscous polymer composition to solidify at body temperature, thereby forming the polymer plug in situ.

2. The method of claim 1, further comprising the step of:
injecting a viscous polymer composition directly into the site.

3. The method of claim 1, wherein the polymer plug is generated in situ by temperature changes, pH changes or ionic interactions.

4. The method of claim 1, further comprising the steps of:
injecting a first composition directly into the site in a mammal; and
injecting a second composition directly into the site in a mammal, wherein the first composition contacts the second composition, thereby forming the viscous polymer composition in situ.

5. The method of claim 1, wherein the method controls bleeding following a catheterization procedure, controls leakage of cerebral spinal fluid following a lumbar puncture, seals a fistula, or controls the flow of serous fluid after a lymphadenectomy.

6. The method of claim 1, wherein the viscous polymer composition comprises at least one optionally purified reverse thermosensitive polymer.

7. The method of claim 18, wherein the at least one optionally purified reverse thermosensitive polymer is a polyoxyalkylene block copolymer.

8. The method of claim 18, wherein the at least one optionally purified reverse thermosensitive polymer is selected from the group consisting of poloxamers and poloxamines.

9. The method of claim 18, wherein the at least one optionally purified reverse thermosensitive polymer is poloxamer 407.

10. The method of claim 18, wherein the at least one optionally purified reverse thermosensitive polymer is selected from the group consisting of purified poloxamers and purified poloxamines.

11. The method of claim 18, wherein the at least one optionally purified reverse thermosensitive polymer is purified poloxamer 407.

12. The method of claim 1, wherein the viscous polymer composition comprises an anionic, cationic, or non-ionically crosslinkable polymer.

13. The method of claim 1, wherein the viscous polymer composition comprises a polymer selected from the group consisting of alginic acid, sodium alginate, potassium alginate, sodium gellan, potassium gellan, carboxy methyl cellulose, hyaluronic acid and polyvinyl alcohol.

14. The method of claim 1, wherein the viscous polymer composition comprises phosphate, citrate, borate, succinate, maleate, adipate, oxalate, calcium, magnesium, barium, or strontium.

15. The method of claim 1, wherein the viscous polymer composition comprises a protein selected from the group consisting of collagen, gelatin, elastin, albumin, protamine, fibrin, fibrinogen, keratin, reelin, and caseine.

16. The method of claim 1, wherein the viscous polymer composition comprises hyaluronic acid, or chitosan.

17. The method of claim 1, wherein the viscous polymer composition comprises alginate, pectin, methylcellulose, or carboxymethylcellulose.

18. The method of claim 1, wherein the viscous polymer composition comprises a crosslinkable polymer.

19. The method of claim 1, wherein the viscous polymer composition is introduced using a syringe, cannula, catheter or percutaneous access device.

20. The method of claim 4, wherein the first composition or the second composition, is introduced using a syringe, cannula, catheter or percutaneous access device.
